SEQUENCE LISTING (13 sequences)

SEQ ID NO: 1 - DNA, 1732 bp, Citrus unshiu
  CDS (50)..(1582)
ggcacgagattgctagctagccaattttagaacaaatcattcgagaataatgggaactgaatctcttgttcatgtcttactagtttcattccccggccatggccacgtaaacccgctcctgaggctcggcagactccttgcttcaaagggtttctttctcaccttgaccacacctgaaagctttggcaaacaaatgagaaaagcgggtaacttcacctacgagcctactccagttggcgacggcttcattcgcttcgaattcttcgaggatggatgggacgaagacgatccaagacgcgaagatcttgaccaatacatggctcaacttgagcttattggcaaacaagtgattccaaaaataatcaagaaaagcgctgaagaatatcgccccgtttcttgcctgatcaataacccatttatcccttgggtctctgatgttgctgaatccctagggcttccgtctgctatgctttgggttcaatcttgtgcttgttttgctgcttattaccattactttcacggtttggttccatttcctagtgaaaaagaacccgaaattgatgttcagttgccgtgcatgccactactgaagcatgatgaaatgcctagcttcttgcatccgtcaactccttatcctttcttgagaagagctattttggggcagtacgaaaatcttggcaagccgttttgcatattgttggacactttctatgagcttgagaaagagattatcgattacatggcaaaaatttgccctattaaacccgtcggccctctgttcaaaaaccctaaagctccaaccttaaccgtccgcgatgactgcatgaaacccgatgaatgcatagactggctcgacaaaaagccaccatcatccgttgtgtacatctctttcggcacggttgtctacttgaagcaagaacaagttgaagaaattggctatgcattgttgaactcggggatttcgttcttgtgggtgatgaagccgccgcctgaagactctggcgttaaaattgttgacctgccagatgggttcttggagaaagttggagataagggcaaagttgtgcaatggagtccacaagaaaaggtgttggctcaccctagtgttgcttgctttgtgactcactgcggctggaactcaaccatggagtcgttggcatcgggggtgccggtgatcaccttcccgcaatggggtgatcaagtaactgatgccatgtatttgtgtgatgtgttcaagaccggtttaagattgtgccgtggagaggcagagaacaggataatttcaagggatgaagtggagaagtgcttgctcgaggccacggccggacctaaggcggtggcgctggaggagaacgcgctgaagtggaagaaggaggcggaggaagctgtggccgatggtggctcgtcggataggaacattcaggctttcgttgatgaagtaagaaggacaagtgtcgagattataaccagcagcaagtcgaagtcaatccacagagttaaggaattagtggagaagacggcaacggcaactgcaaatgacaaggtagaattggtggagtcacgacggacacgtgtacagtattgattggaagttctgactcaaagcacttgtcgagttgtcgtaaataaaatgtttcataataatcatattttgcactactttataattacgtgatgtttttatcttaatgtacttatctattccctttcaaaataaaaaaaaaaaaaaaaa

SEQ ID NO: 2 - protein, 511 aa, Citrus unshiu
MGTESLVHVLLVSFPGHGHVNPLLRLGRLLASKGFFLTLTTPESFGKQMRKAGNFTYEPTPVGDGFIRFEFFEDGWDEDDPRREDLDQYMAQLELIGKQVIPKIIKKSAEEYRPVSCLINNPFIPWVSDVAESLGLPSAMLWVQSCACFAAYYHYFHGLVPFPSEKEPEIDVQLPCMPLLKHDEMPSFLHPSTPYPFLRRAILGQYENLGKPFCILLDTFYELEKEIIDYMAKICPIKPVGPLFKNPKAPTLTVRDDCMKPDECIDWLDKKPPSSVVYISFGTVVYLKQEQVEEIGYALLNSGISFLWVMKPPPEDSGVKIVDLPDGFLEKVGDKGKVVQWSPQEKVLAHPSVACFVTHCGWNSTMESLASGVPVITFPQWGDQVTDAMYLCDVFKTGLRLCRGEAENRIISRDEVEKCLLEATAGPKAVALEENALKWKKEAEEAVADGGSSDRNIQAFVDEVRRTSVEIITSSKSKSIHRVKELVEKTATATANDKVELVESRRTRVQY

SEQ ID NO: 3 - protein, 13 aa, Citrus unshiu
GTESLVHVLLVSF

SEQ ID NO: 4 - protein, 14 aa, Citrus unshiu
AGNFTYEPTPVGDG

SEQ ID NO: 5 - protein, 13 aa, Citrus unshiu
AEEAVADGGSSDR

SEQ ID NO: 6 - DNA, 23 bp, Artificial Sequence
  misc_feature (3)..(3): “n” is a, c, g or t
ggnacngarwsnytngtncaygt

SEQ ID NO: 7 - DNA, 17 bp, Artificial Sequence
  misc_feature: Description of Artificial SequenceDesigned oligonucleotide based on a partial amino acid sequence of UDP-D-glucoselimonoid gluco syltransferase from Citrus unshi
ccrtcngcnacngcytc

SEQ ID NO: 8 - DNA, 45 bp, Artificial Sequence
  misc_feature: Description of Artificial SequencePrimer
aactggaagaattcgcggccgcaggaatttttttttttttttttt

SEQ ID NO: 9 - DNA, 21 bp, Artificial Sequence
  misc_feature: Description of Artificial SequenceNot I adapter sequence
tggaagaattcgcggccgcag

SEQ ID NO: 10 - DNA, 1536 bp, Citrus unshiu
  CDS (1)..(1533)
atgggaactgaatctcttgttcatgtcttactagtttcattccccggccatggccacgtaaacccgctcctgaggctcggccgactccttgcttcaaagggtttctttctcaccttgaccacacctgaaagctttggcaaacaaatgagaaaagcgggtaacttcacctacgagcctactccagttggcgacggcttcattcgcttcgaattcttcgaggatggatgggacgaagacgatccaagacgcggagatcttgaccaatacatggctcaacttgagcttattggcaaacaagtgattccaaaaataatcaagaaaagcgctgatgaatatcgccccgtttcttgcctgatcaataacccatttatcccttgggtctctgatgttgctgaatccctagggcttccgtctgctatgctttgggttcaatcttgtgcttgttttgctgcttattaccattactttcacggtttggttccatttcctagtgaaaaagaacccgaaattgatgttcagttgccgtgcatgccactactgaagcatgatgaagtgcctagcttcttgcatccgtcaactccttatcctttcttgagaagagctattttggggcagtacgagaatcttggcaagccgttttgcatattgttggacactttctatgagcttgagaaagagattatcgatcacatggcaaaaatttgccctattaaacccgtcggccctctgttcaaaaaccctaaagctccaaccttaaccatccgcgatgactgcatgaaacccgatgaatgcatagactggctcgacaaaaagccaccatcatccgttgtgtacatctctttcggcacggttgtctacttgaagcaagaacaagttgaagaaattggctatgcattgttgaactcggggatttcgttcttgtgggtgatgaagccgccgtctgaagactctggcgttaaaattgttggcctgccagatgggttcttggagaaagttggagataagggcaaagttgtgcaatggagtccacaagaaaaggtgttggctcaccctagtgttgcttgctttgtgactcactgcggctggaactcaaccatggagtcgttggcatcgggggtgccggtgatcaccttcccgcaatggggtgatcaagtaactgatgccatgtatttgtgtgatgtgttcaagaccggtttaagattgtgccgtggacaggcagagaacaggataatttcaagggatgaagtggagaagtgcttgctcgaggccacggccggacctaaggcggcggagctgaaggagaacgcgctgaagtggaagaaggaggcggaggaagctgtggccgatggtggctcgtcggataggaacattcaggctttcgttgatgaagtaagaaggagaagtgtcgagatcataaccagcagcaagtcgaagtcaatccacagagttaaggaattagtggagaagacggcaacggcaactgcaaatgacaaggtagaattggtggagtcacgacggacacgtgtacagtattga

SEQ ID NO: 11 - protein, 511 aa, Citrus unshiu
MGTESLVHVLLVSFPGHGHVNPLLRLGRLLASKGFFLTLTTPESFGKQMRKAGNFTYEPTPVGDGFIRFEFFEDGWDEDDPRRGDLDQYMAQLELIGKQVIPKIIKKSADEYRPVSCLINNPFIPWVSDVAESLGLPSAMLWVQSCACFAAYYHYFHGLVPFPSEKEPEIDVQLPCMPLLKHDEVPSFLHPSTPYPFLRRAILGQYENLGKPFCILLDTFYELEKEIIDHMAKICPIKPVGPLFKNPKAPTLTIRDDCMKPDECIDWLDKKPPSSVVYISFGTVVYLKQEQVEEIGYALLNSGISFLWVMKPPSEDSGVKIVGLPDGFLEKVGDKGKVVQWSPQEKVLAHPSVACFVTHCGWNSTMESLASGVPVITFPQWGDQVTDAMYLCDVFKTGLRLCRGQAENRIISRDEVEKCLLEATAGPKAAELKENALKWKKEAEEAVADGGSSDRNIQAFVDEVRRRSVEIITSSKSKSIHRVKELVEKTATATANDKVELVESRRTRVQY

SEQ ID NO: 12 - DNA, 24 bp, Artificial Sequence
  misc_feature: Description of Artificial SequencePrimer
atgggaactgaatctcttgttcat

SEQ ID NO: 13 - DNA, 24 bp, Artificial Sequence
  misc_feature: Description of Artificial SequencePrimer
tcaatactgtacacgtgtccgtcg

(19) United States
(12) Patent Application Publication
(10) Pub. No.: US 2002/0106769 A1
(43) Pub. Date: Aug. 8, 2002

(54) UDP-D-glucose: limonoid glucosyltransferase

(76) Inventors: Mitsuo Omura, Shizuoka-Ken (JP); Tomoko Inagaki, Shizuoka (JP); Ryoji Matsumoto, Nagasaki (JP); Takaya Moriguchi, Ibaraki (JP); Shin Hasegawa, Albany, CA (US); Charles G. Suhayda, Pasadena, CA (US)

Correspondence Address: USDA-ARS-OFFICE OF TECHNOLOGY TRANSFER, PATENT ADVISORS OFFICE, WESTERN REGIONAL RESEARCH CENTER, 800 BUCHANAN ST, ALBANY, CA, 94710, US

(21) Appl. No.: 09/773,882

(22) Filed: Jan. 31, 2001

(30) Foreign Application Priority Data
Jan. 31, 2000 (JP) 2000-021179

(51) Int. Cl. C12N 9/10

(52) U.S. Cl. 435/193

(57) ABSTRACT

To provide UDP-D-glucose:limonoid glucosyltransferase, and DNA coding for the enzyme.

The recombinant protein of (a) or (b) below:

(a) protein comprising the amino acid sequence in Sequence ID No. 2;

(b) protein comprising amino acid sequence in Sequence ID No. 2 with one or more amino acid deletions, substitutions or additions, and having UDP-D glucose:limonoid glucosyltransferase activity.

The recombinant protein of (a) or (b) below:

(a) protein comprising the amino acid sequence in Sequence ID No. 2;

(b) protein comprising amino acid sequence in Sequence ID No. 2 with one or more amino acid deletions, substitutions or additions, and having UDP-D-glucose:limonoid glucosyltransferase activity.

The recombinant protein of (a) or (b) below:

(a) protein comprising the amino acid sequence in Sequence ID No. 11;

(b) protein comprising amino acid sequence in Sequence ID No.11 with one or more amino acid deletions, substitutions or additions, and having UDP-D-glucose:limonoid glucosyltransferase activity.

DNA coding for the recombinant protein of (a) or (b) below:

(a) protein comprising the amino acid sequence in Sequence ID No. 2;

(b) protein comprising amino acid sequence in Sequence ID No. 2 with one or more amino acid deletions, substitutions or additions, and having UDP-D-glucose:limonoid glucosyltransferase activity.

DNA according to claim 3, comprising the base sequence in Sequence ID No. 1.

DNA coding for the recombinant protein of (a) or (b) below:

(a) protein comprising the amino acid sequence in Sequence ID No. 11;

(b) protein comprising amino acid sequence in Sequence ID No. 11 with one or more amino acid deletions, substitutions or additions, and having UDP-D-glucose:limonoid glucosyltransferase activity.

DNA according to claim 5, comprising the base sequence in Sequence ID No. 10.

Recombinant vectors comprising the DNA in any of claims 3 through 6.

Transformants which have been transformed by a recombinant vector according to claim 7.

A method for producing UDP-D-glucose:limonoid glucosyltransferase, characterized in that transformants according to claim 8 are cultured in media, and UDP-D-glucose:limonoid glucosyltransferase is harvested from the resulting culture.

A method for producing limonoid glucosides, characterized in that transformants according to claim 8 are cultured in media, and limonoid glucosides are extracted from the resulting culture.

### DETAILED DESCRIPTION OF THE INVENTION

1. Field of Industrial Application

The present invention relates to UDP-D-glucose:limonoid glucosyltransferase, DNA coding for UDP-D-glucose:limonoid glucosyltransferase, recombinant vectors comprising such DNA, transformants which have been transformed by such vectors, and methods for producing UDP-D-glucose:limonoid glucosyltransferase and limonoid glucosides.

2. Prior Art

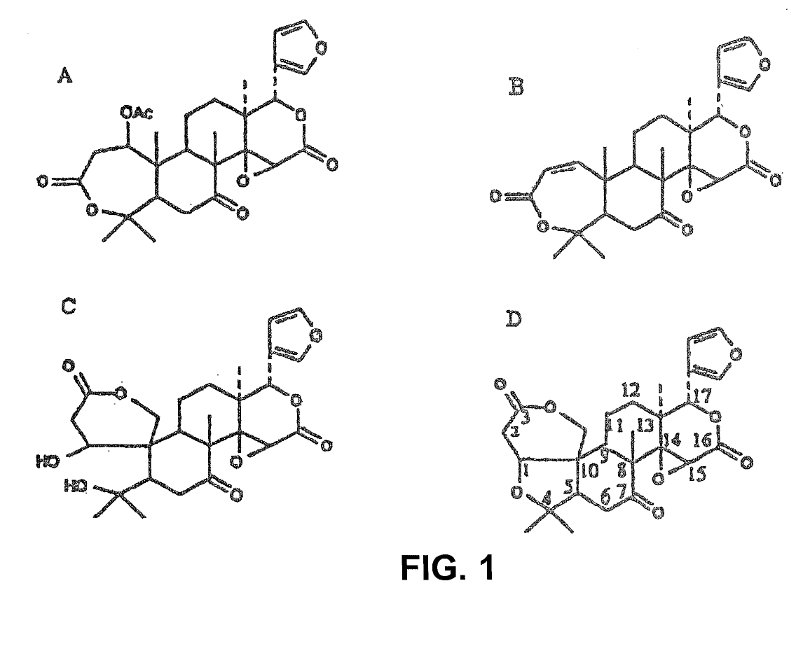

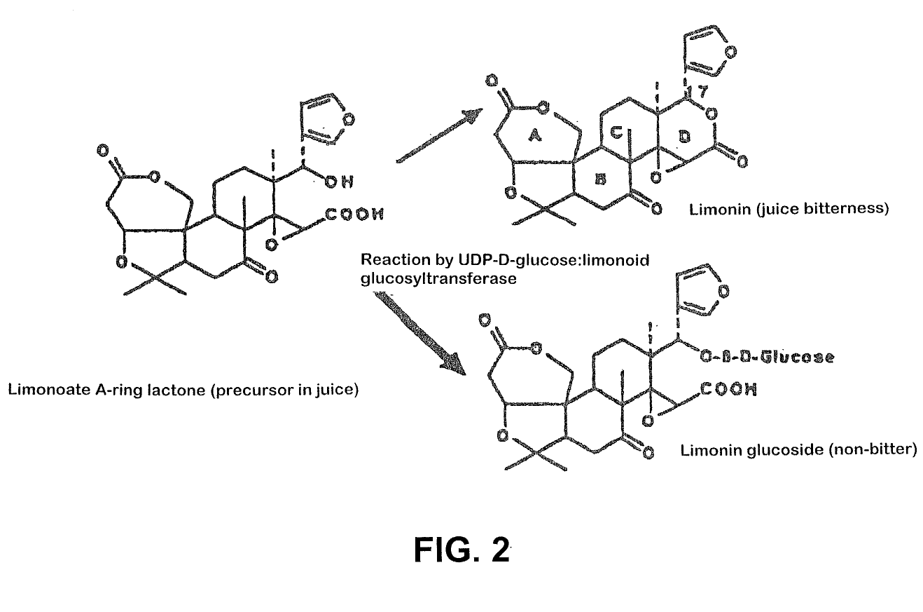

Triterpenoids, which are biosynthetic products of Rutaceae and Meliacea plants, include the group of compounds known as limonoids. The major limonoids in citrus are limonin, nomilin, ichangin, and obacunone (FIG. 1). In plant materials such as fruit juices, these substances occur with the D-ring open, and in the form of limonoate A-ring lactone (LARL) in the case of limonins (FIG. 2). The D-rings form lactones under acidic conditions, giving these substances their bitter taste. The reaction is accelerated by limonoid D-ring lactone hydrolase.

In the case of A-ring lactone limonoids, non-bitter tasting limonoid glucosides are formed with the addition of one glucose to the D-ring of these substances upon maturity in citrus fruits (FIG. 2). 17 types of citrus limonoid glucosides, such as limonin glucoside, nomilin glucoside, and obacunone glucoside, have been isolated from citrus and its hybrids.

Mandarin juice has a high limonoid:limonoid glucoside ratio of 1:150, with few bitter-tasting aglycones lacking the addition of glucose. Oranges and grapefruit, on the other hand, contain enough limonoid aglycones to result in a bitter taste. Some way of eliminating the bitterness is needed in the latter case. Currently employed methods include industrial methods such as the adsorption of limonoid aglycones using ion exchange resins after juicing. However, substances other than bitter components are also adsorbed in such methods, resulting in juice of lower quality and a shorter shelf life.

The production of limonoid glucosides is known to be catalyzed by UDP-D-glucose:limonoid glucosyltransferase. Limonoid glucoside production has been confirmed with the extracted enzyme. The gene coding for the enzyme protein has remained unknown, however.

### PROBLEMS WHICH THE INVENTION IS INTENDED TO SOLVE

An object of the present invention is to provide UDP-D-glucose:limonoid glucosyltransferase and a gene coding for that enzyme.

### MEANS USED TO SOLVE THE ABOVE-MENTIONED PROBLEMS

As a result of extensive research to overcome the drawbacks described above, the inventors perfected the present invention upon successfully isolating DNA coding for UDP-D-glucose:limonoid glucosyltransferase from a citrus cDNA library.

That is, the present invention provides the recombinant protein of (a) or (b) below:

(a) protein comprising the amino acid sequence in Sequence ID No. 2;

(b) protein comprising amino acid sequence in Sequence ID No. 2 with one or more amino acid deletions, substitutions or additions, and having UDP-D-glucose:limonoid glucosyltransferase activity.

As used herein, “UDP-D-glucose:limonoid glucosyltransferase activity” means the activity associated with the catalysis producing limonoid glucosides such as nomilin glucoside and limonin glucoside from limonoids such as nomilin and limonin.

The present invention also provides the recombinant protein of (a) or (b) below:

(a) protein comprising the amino acid sequence in Sequence ID No. 11;

(b) protein comprising amino acid sequence in Sequence ID No. 11 with one or more amino acid deletions, substitutions or additions, and having UDP-D-glucose:limonoid glucosyltransferase activity.

The present invention furthermore provides DNA coding for the recombinant protein of (a) or (b) below:

(a) protein comprising the amino acid sequence in Sequence ID No. 2;

(b) protein comprising amino acid sequence in Sequence ID No. 2 with one or more amino acid deletions, substitutions or additions, and having UDP-D-glucose:limonoid glucosyltransferase activity.

In addition, the present invention provides DNA coding for UDP-D-glucose:limonoid glucosyltransferase, comprising the base sequence in Sequence ID No. 1.

The present invention furthermore provides DNA coding for the recombinant protein of (a) or (b) below:

(a) protein comprising the amino acid sequence in Sequence ID No. 11;

(b) protein comprising amino acid sequence in Sequence ID No. 11 with one or more amino acid deletions, substitutions or additions, and having UDP-D-glucose:limonoid glucosyltransferase activity.

In addition, the present invention provides DNA coding for UDP-D-glucose:limonoid glucosyltransferase, comprising the base sequence in Sequence ID No. 10.

The present invention also provides recombinant vectors such DNA.

The present invention also provides transformants which have been transformed by such recombinant vectors.

The present invention furthermore provides a method for producing UDP-D-glucose:limonoid glucosyltransferase, characterized in that transformants according to claim 8 are cultured in media, and UDP-D-glucose:limonoid glucosyltransferase is harvested from the resulting culture.

In addition, the present invention provides a method for producing limonoid glucosides, characterized in that transformants according to claim 8 are cultured in media, and limonoid glucosides are extracted from the resulting culture.

### Embodiments of the Invention

The present invention is illustrated in greater detail below.

1. Cloning of DNA coding for UDP-D-glucose:limonoid Glucosyltransferase

DNA coding for UDP-D-glucose:limonoid glucosyltransferase can be isolated from a cDNA library by determining partial amino acid sequences of the enzyme, and by preparing a pair of primers based on the sequences to prepare partial cDNA of the enzyme for use as a probe. The pair of primers prepared on the basis of the resulting DNA sequences can also be used to clone DNA coding for the enzyme from the genome.

1) Partial sequencing of amino acid sequences of UDP-D-glucose:limonoid glucosyltransferase

The aforementioned partial amino acid sequences can be determined by extracting and purifying UDP-D-glucose:limonoid glucosyltransferase from plants, and by partially sequencing the proteins that are obtained.

The plants that are extracted are limited only to the extent that they produce the enzyme, although citrus is preferred, especially sweet oranges, and ideally naval oranges (Citrus sinensis Osbeck var. brasiliensis Tanaka). The location at which the plants are to be extracted is limited only to the extent that the location includes the enzyme that is produced, preferably the peel, and ideally the albedo of the peel. Any known method used in the art can be used for extraction.

Any known method for purifying proteins can be used. The resulting purified enzyme should be further isolated by electrophoresis or the like. Protein of between 56 and 58 kD can be isolated by SDS-PAGE, for example.

At least two partial amino acid sequences are determined for the enzyme protein thus obtained. Although the portions that are sequenced are not particularly limited, amino-terminal sequences and interior sequences are preferred. An interior sequence can be determined, for example, using a protease or the like to partially degrade the enzyme protein, and by then sequencing the resulting fragments. Such partial amino acid sequencing can be done by a common method in the art, such as Edman degradation.

The partially determined amino acid sequences are not limited to any particular sequence. The following are examples.

Amino-terminal Sequence:

| | |
|---|---|
| i) GTESLVHVLLVSF | (Sequence ID No. 3) |

Interior Partial Sequence:

| | |
|---|---|
| ii) AGNFTYEPTPVGDG and | (Sequence ID No. 4) |
| iii) AEEAVADGGSSDR | (Sequence ID No. 5) |

2) Preparation of Primers and Preparation of Partial cDNA by RT-PCR

A pair of primers can be designed and synthesized based on the two amino acid sequences obtained in section 1), and these can be used in RT-PCR to prepare partial cDNA.

The primers can be designed by designing the sense primer from the amino acid sequence closer to the amino-terminal among the two aforementioned amino acid sequences, and by designing the antisense primer from the one closer to the carboxyl terminal. Those having ordinary skill in the art can appropriately design such primers by designing universal primers in consideration of the degeneracy of the codons corresponding to the amino acids. The sequences of the primers that are thus designed are not limited to any particular sequences. The following sequences, for example, can be produced by designing the sense primer on the basis of the amino acid sequence of i) (Sequence ID No. 3) in section 1) above, and by designing the antisense primer on the basis of the amino acid sequence of iii) (Sequence ID No. 5).

Sense Primer (Llg):

(Sequence ID No. 6)

GGNACNGA(a/g)(a/t)(g/c)N(c/t)TNGTNCA(c/t)GT

Antisense Primer (lgt14pr):

| | |
|---|---|
| CC(a/g)TCNGCNACNGC(t/c)TC | (Sequence ID No. 7) |

The primers that have thus been designed can be synthesized by methods known to those having ordinary skill in the art as methods for synthesizing oligonucleotides.

The synthesized sense and antisense primers are then used in RT-PCR. RT-PCR (reverse transcription-PCR) is a method in which reverse transcriptase is used in the pre-synthesis of DNA using RNA template, and the synthesized DNA is again used as template for PCR.

Plant-derived mRNA can be used as the RNA for the aforementioned template. The plant may be any that produces such enzymes, and is not particularly limited. Citrus is preferred, especially mandarins, and ideally Citrus unshiu Marc. The portion of the plant that is used to prepare the mRNA is limited only to the extent that mRNA is present in the site used. The peel is preferred, especially the albedo of the peel. Methods known in the art can be used to prepare the mRNA, and are not particularly limited. For example, total RNA can be isolated using the SDS-phenol method or the like from the organs or tissue of the aforementioned plants (such as the albedo, flavedo, and leaves when the plant is citrus), and the mRNA can be prepared with an affinity column using oligo dT-cellulose or the like.

DNA can be synthesized with reverse transcriptase by a common method. For example, the selection of the reagents in addition to the reverse transcriptase, the preparation of the reaction mixture, the establishment of the reaction conditions such as the reaction temperature and reaction time, and so forth can be suitably managed by one having ordinary skill in the art. The DNA may also be synthesized using a commercially available kit (such as First Strand cDNA synthesis kit by Pharmacia).

PCR using the synthesized DNA as template can be carried out by a common method using the primers synthesized above. For example, the selection of the reagents in addition to the DNA polymerase, the preparation of the reaction mixture, the establishment of the reaction conditions such as the reaction temperature, reaction time, and number of cycles, and so forth can be suitably managed by one having ordinary skill in the art. PCR may also be carried out using a commercially available kit (such as Ampli Taq Gold DNA polymerase, by Perkin Elmer Applied Biosystems).

3) Preparation of cDNA Library

The plant-derived cDNA library used to prepare the mRNA in section 2) above can be prepared in the following manner in order to obtain the full-length cDNA of the UDP-D-glucose:limonoid glucosyltransferase using as probe the partial cDNA obtained in section 2) above.

Total RNA is first extracted from the plant to then isolate the mRNA. The plant used to extract the total RNA is not particularly limited, provided that it produces the enzyme. Citrus is preferred, especially mandarins, and ideally Citrus unshiu Marc. The portion of the plant that is extracted is limited only to the extent that mRNA of the enzyme is present in the site used. The peel is preferred, especially the albedo of the peel. Methods known in the art can be used for the extraction, and are not particularly limited. For example, total RNA can be extracted using the SDS-phenol method or the like from the organs or tissue of the aforementioned plants (such as the albedo, flavedo, and leaves when the plant is citrus). The mRNA can be isolated by a common method in the art using the extracted total RNA. This can be managed, for example, with an affinity column using oligo dT-cellulose or the like.

The resulting mRNA is then used as template to synthesize single-stranded cDNA with reverse transcriptase, and double-stranded cDNA is then synthesized from the single-stranded cDNA. The single-stranded cDNA can be synthesized by a common method using suitable reverse transcriptase and primers. Reverse transcriptase derived from Moloney Murine Leukemia Virus (MMLV) is an example of suitable reverse transcriptase. Oligo dT primers capable of hybridizing with the poly A chain of mRNA are preferably used for primers. The double-stranded cDNA can be synthesized from the single-stranded cDNA by a common method using DNA polymerase. A commercially available kit (cDNA synthesis kit, by Pharmacia) can be used until the synthesis of the double-stranded cDNA from the mRNA.

The resulting double-stranded cDNA is ligated using ligase to a suitable plasmid or phage vector, and the resulting recombinant DNA is used to infect or transform E. coli so as to obtain a cDNA library. This series of operations is done in the usual manner, and can be readily managed by one having ordinary skill in the art.

4) Isolation of cDNA Clones of the UDP-D-glucose:limonoid Glucosyltransferase Gene from the cDNA Library

The full-length cDNA of the UDP-D-glucose:limonoid glucosyltransferase gene is screened from the resulting cDNA library by hybridization using the partial cDNA obtained in section 2) above as probe. Hybridization can be done by a common method such as plaque hybridization or colony hybridization, and is not limited to any specific procedure. Hybridization can be managed, for example, using a commercially available kit such as the ECL nucleic acid labeling and detection system (Pharmacia) or DIG DNA labeling and detection kit (Boehringer Mannheim). The cDNA clones can be isolated by a common method known to those having ordinary skill in the art from the plaques, colonies, or the like selected by the aforementioned screening.

5) Base Sequencing

The base sequences of the cDNA clones obtained in section 4) are then determined. The base sequences can be determined by a common procedure such as the Maxam-Gilbert method or the dideoxy method, but sequencing is usually done with an automated base sequencer. The base sequence in Sequence ID No. 1 is an example of a base sequence determined in this manner.

6) Isolation of Genomic Clones by PCR

A pair of primers can be designed on the basis of the base sequences of the cDNA obtained in section 5) so as to amplify and clone the genomic DNA. Base sequencing can be done in the same manner as in section 5).

The primers can be designed by designing the sense primer from around the 5′ end of Sequence ID No. 1 and by designing the antisense primer from around the 3′ end. The sequences of the primers designed in this manner are not limited to any particular sequences, but to obtain genomic clones covering the entire code sequence, the sense primer can begin from the translation initiation codon at 50 (Sequence ID No. 12) in Sequence ID No. 50, and the antisense primer can begin from the stop codon at 1585 (Sequence ID No. 13).

(Sequence ID No. 12)

sense primer: ATGGGAACTGAATCTCTTGTTCAT

(Sequence ID No.13)

antisense primer: TCAATACTGTACACGTGTCCGTCG

The primers designed in this manner can be synthesized by methods known to those having ordinary skill in the art as methods for synthesizing oligonucleotides.

Total DNA derived from Rutaceae plants producing limonoid glucosides can be used as the genomic DNA serving as template in PCR amplification. The plant is preferably citrus, ideally Citrus unshiu Marc. The portion of the plant that is used to prepare the DNA is limited only to the extent that the DNA is present in the site used. The leaves are preferred, especially mature leaves without mid ribs. Common methods in the art can be used to prepare the DNA, and are not particularly limited. For example, total DNA can be extracted and purified by the method of Dellaporta et al (Plant Molecular Biology Reporters, Vol.1, pp. 19-21(1983)).

PCR using the extracted DNA as template can be carried out in the usual manner. For example, the selection of the reagents in addition to the DNA polymerase, the preparation of the reaction mixture, the establishment of the reaction conditions such as the reaction temperature, reaction time, and number of cycles, and so forth can be suitably managed by one having ordinary skill in the art. PCR may also be carried out using a commercially available kit (such as Ampli Taq Gold, by Perkin Elmer Applied Biosystems).

The double-stranded DNA obtained in this manner is ligated using ligase to a suitable plasmid, such as pCRII (Invitrogen), the resulting recombinant DNA is used for transformation with E. coli so as to obtain genomic clones, and the base sequences are determined in the manner described above in section 5). The base sequence in Sequence ID No. 10 is an example of a base sequence determined in this manner.

7) DNA and Protein of the Invention

The DNA of the present invention is DNA comprising the base sequence in Sequence ID No. 1 or 10 determined in the manner described above. The proteins of the present invention comprise the amino acid sequence in Sequence ID No. 2 or 11 deduced from the above base sequences, respectively. These proteins function as UDP-D-glucose:limonoid glucosyltransferase. The amino acid sequences of the proteins of the present invention are not limited to the sequences in Sequence ID No. 2 and 11. Mutations involving one or more amino acid deletions, substitutions, additions or the like to the above amino acid sequences may be produced, providing that the UDP-D-glucose:limonoid glucosyltransferase activity is preserved. For example, the proteins of the present invention include deletions of the methionine at 1 in the amino acid sequences in Sequence ID No. 2 or 11. The base sequence of the DNA in the present invention is also not limited to the sequences in Sequence ID No. 1 and 10. The DNA of the present invention also includes any coding for proteins of the present invention such as the above.

Once the base sequence of the DNA in the present invention is determined, the DNA of the present invention can subsequently be obtained by a chemical reaction or by hybridization using as probe any DNA fragments having said base sequences. Such chemical reactions and hybridization can be managed by methods known to those having ordinary skill in the art.

2. Preparation or Recombinant Vectors and Transformants

1) Preparation of Recombinant Vectors

Recombinant vectors of the present invention can be prepared by ligating (inserting) the DNA of the present invention to suitable vectors.

Vectors for insertion of the DNA of the present invention are not particularly limited, provided that they are replicable in hosts. Examples include plasmid DNA and phage DNA. Plasmid DNA can be prepared by alkali extraction from microbes such as E. coli or Agrobacterium (H. C. Birnboim and J. Doly, Nucleic Acid Res. 7:1513 (1979)) or by modified methods thereof. Commercially available plasmids such as pBluescript II SK+ (Stratagene), pUC118 (Takara Shunzo), and pGEX4T-1 (Pharmacia) may also be used. Such plasmids will preferably include an ampicillin resistance gene, kanamycin resistance gene, chloramphenicol resistance gene, or the like. Examples of phage DNA include M13mp18 and M13mp19.

To insert the DNA of the present invention into a vector, purified DNA is digested, for example, with suitable restriction enzymes, inserted at a suitable multicloning site or restriction enzyme site of the vector DNA, and ligated to the vector. Such procedures are commonly employed in the art, and can be readily managed according to the specific base sequence of the DNA to be inserted by those having ordinary skill in the art.

The DNA of the present invention must be incorporated in a vector in such a way as to ensure that the function of the DNA is brought out. Terminators, ribosome binding sequences, and the like may be incorporated, in addition to promoters and the DNA of the present invention, in the vectors of the present invention. Such procedures are commonly employed in the art and can be readily managed by those having ordinary skill in the art.

2) Preparation of Transformants

The transformants of the present invention can be obtained by introducing the recombinant vector of the present invention into a host in such a way that allows the target gene to be expressed. The host is not particularly limited, provided that it is capable of expressing the DNA of the present invention. Examples include bacterial cells, yeasts, and animal and plant cells. Examples of bacterial cells include Escherichia such as E. coli (Escherichia coli), and Subtilis such as Bacillus subtilis. Examples of yeast include Saccharomyces cerevisiae. Examples of animal and plant cells include CHO cells and tobacco BY-2 cells.

When bacterial cells such as E. coli are used as the host, the recombinant vectors of the present invention should be autonomously replicable in such hosts, and should at the same time comprise a promoter, ribosome-binding sequence, the DNA of the present invention, and a translation termination sequence. Examples of expression vectors suitable for such purposes include the pBluescript II vector, pET vector (Stratagene), and pGEX4T-1 (Pharmacia). Any promoter that can be expressed in hosts such as E. coli can be used. Examples of promoters that can be used include promoters derived from E. coli or phages, such as the Trp promoter, lac promoter, PL promoter, and PR promoter. Recombinant vectors of the present invention may also include a gene for controlling the promoter.

The recombinant vector of the present invention may be introduced into bacterial cells by any method allowing DNA to be introduced into such cells. Examples include the method using calcium ions (Proc. Natl. Acad. Sci., USA 69:2110-2114 (1972)).

Examples of expression vectors that can be used when the host is a yeast include YEp13, YEp24, and YCp50. The promoter used in such cases may be any that can be expressed in yeast. Examples include the gal1 promoter, gal10 promoter, the heat sink protein promoter, and the MFα1 promoter.

Any method allowing DNA to be introduced into yeasts can be used to introduce the recombinant vector of the present invention into yeasts. Examples include electroporation (Methods. Enzymol., 194:182-187 (1990), the spheroplast method (Proc. Natl. Acad. Sci. USA, 84:1929-1933 (1978)), and the lithium acetate method (J. Bacteriol., 153:163-168 (1983)).

The pcDNAI/Amp expression vector (Invitrogen) may be used when the host is an animal cell. The initial gene promoter of human cytomegalovirus may be used as a promoter at such times.

Any method allowing DNA to be introduced to animal cells can be used to introduce the recombinant vector of the present invention to animal cells. Examples include electroporation, the calcium phosphate method, and lipofection.

The pBI121 (Clontech) expression vector can be used for plant cell hosts. The cauliflower mosaic virus 35S protein gene promoter may be used as the promoter in such cases.

Any method allowing DNA to be introduced to plant cells can be used to introduce the recombinant vector of the present invention to plant cells. Examples include the Agrobacterium method, electroporation, and bombardment.

The recombinant vector pCitLGT of the present invention (containing the coding sequence of the base sequence in Sequence ID No. 1) and pCitLGT-2 (containing the base sequence in Sequence ID No. 10) have been introduced to E. coli, and have been registered as FERM P-17065 (CitLGT) and FERM P-17537 (CitLGT-2) at the Life Sciences Research Institute of the Ministry of Industrial Technology (1-1-3 Higashi, Tsukuba City, Ibaraki Prefecture).

3. Production of UDP-D-glucose:limonoid glucosyltransferase

The UDP-D-glucose:limonoid glucosyltransferase of the present invention can be harvested from cultures obtained by culturing the aforementioned transformants in media.

The transformants in the present invention are cultured in media by methods commonly employed for the culture of hosts. The medium used for culturing transformants obtained using microbes such as E. coli or yeast as the host can be any natural or synthetic medium, provided that it contains microbially degradable carbon sources, nitrogen sources, inorganic salts, and the like, allowing the transformants to be cultured efficiently.

Examples of carbon sources include carbohydrates such as glucose, fructose, sucrose, and starch, organic acids such as acetic acid and propionic acid, and alcohols such as ethanol and propanol.

Examples of nitrogen sources include ammonia salts of inorganic or organic acids, such as ammonia, ammonium chloride, ammonium sulfate, ammonium acetate, and ammonium phosphate, or other nitrogenous compounds, peptone, meat extracts, corn steep liquor, and the like.

Examples of inorganic acid salts include monobasic potassium phosphate, dibasic potassium phosphate, magnesium phosphate, magnesium sulfate, sodium chloride, ferrous sulfate, manganese sulfate, copper sulfate, and calcium carbonate.

Transformants obtained with microbial hosts are normally cultured for 48 to 60 hours at about 28° C. under aerobic conditions such as shaking culture or aerated spin culture. The pH is maintained at between 7.0 and 7.5 during the culture. The pH is adjusted using an inorganic acid, organic acid, alkaline solution, or the like. Antibiotics such as ampicillin or tetracycline may be added as needed during the culture.

An inducer may be added as needed to the medium during the culture of microbes transformed with expression vectors having an inductive promoter. Isopropyl-β-D-thiogalactosylpyranoside (IPTG) or the like may be added to the medium during the culture of microbes transformed with an expression vector having a Lac promoter, for example, and indoleacrylic acid or the like may be added to the medium during the culture of microbes transformed with an expression vector having a Trp promoter.

Commonly used RPMI 1640 medium, DMEM medium, or such media supplemented with fetal calf serum or the like may be used as the medium to culture transformants obtained with animal cell hosts.

Commonly used Murashige and Skoog (MS) media may be used as the medium for culturing transformants obtained with plant cell hosts.

Transformants obtained with animal cell hosts are normally cultured for 1 to 2 days at about 37° C. in the presence of 5% CO₂. Antibiotics such as kanamycin and penicillin may be added to the medium as needed during culture.

The UDP-D-glucose:limonoid glucosyltransferase of the present invention can then be harvested from the resulting culture. When the enzyme is produced in cells or bacteria, the cells or bacteria can be ruptured or the like to harvest the enzyme. When the enzyme is extracellularly produced, the culture broth can be used as such, or the bacteria or cells can be centrifuged off before the enzyme is harvested. The enzyme can be harvested by a biochemical method commonly used in the isolation and purification of proteins, such as ammonium sulfate precipitation, affinity chromatography, or ion exchange chromatography, either alone or in combination.

Common enzymological reactions, electrophoresis such as SDS-PAGE, immunological methods such as antigen-antibody reactions, and the like can be used to confirm that the resulting proteins are UDP-D-glucose:limonoid glucosyltransferase.

The UDP-D-glucose:limonoid glucosyltransferase of the present invention is important for catalyzing the production of essential and important substances for preserving the quality and function of citrus fruits.

4. Production of Limonoid Glucosides

Limonoid glucosides can be produced in the present invention in the same manner as procedures for purifying the UDP-D-glucose:limonoid glucosyltransferase. That is, the aforementioned transformants are cultured in medium, and the limonoid glucosides are extracted from the culture. The culturing methods are the same as those in the section entitled “3. Production of UDP-D-glucose:limonoid glucosyltransferase.”

Following the culture, the bacteria or cells are centrifuged off, and the limonoid glucosides can be extracted and purified by HPLC or with the use of a column such as XAD or DEAE-sepharose resin, etc. Such extraction and purification are commonly carried out in the art and can be readily managed by those having ordinary skill in the art. In cases where fractions are produced from the eluate obtained by the aforementioned columns or HPLC, the concentration of the limonoid glucosides in the fractions can be measured to harvest fractions meeting the prescribed concentration. The concentration of limonoid glucosides can be determined by methods known in the art. For example, the fractions can be adsorbed on Sep-Pak, and the methanol eluate can be subjected to HPLC using a C18 reverse phase column (Ozaki et al, J. Food Sci. 60, 186-189 & 194 (1995)).

NMR, mass spectrometry, HPLC, thin layer chromatography, and the like can be used to verify that the substance ultimately extracted is a limonoid glucoside. Those having ordinary skill in the art can identify limonoid glucosides in such methods based on the chemical structure or other properties. For example, the known analytical data obtained by such methods can be compared with experimental data. When known analytical data cannot be used, the analytical data obtained for limonoid glucosides prepared by chemical synthesis may be used.

### EXAMPLES

The present invention is illustrated in further detail in the following examples. The technical scope of the present invention is not limited by these examples, however.

### Example 1

### Cloning of cDNA Coding for UDP-D-glucose:limonoid Glucosyltransferase

1) Partial Amino Acid Sequencing of UDP-D-glucose:limonoid Glucosyltransferase

1000 mL of 50 mM Tris hydrochloric acid buffer (pH 8.0, supplemented with 5 mM DTT, 100 mM KCl, 0.5% PVPP, and 0.5 mM PMSF) was added to 250 g albedo from the peel of naval oranges (Citrus sinensis Osbeck var. brasiliensis Tanaka), the albedo was ground, the ground material was centrifuged, and the supernatant was recovered. Fractions precipitated with a 40 to 80% concentration of ammonium sulfate were recovered from the supernatant and dissolved in 10 mL of test buffer (20 mM MES-KOH pH 6.8, supplemented with 1 mM DTT and 50 μM MnCl₂), giving a crude enzyme solution. 2.5 mL of the crude enzyme solution was desalinated on a PD-10 column (Pharmacia), eluted with 3.5 mL test buffer, then adsorbed to an affinity column with a 75 mL volume bed using UDP-glucuronic acid agarose beads (Sigma) as the affinity support, and eluted with 10 mM UDP-glucose.

The partially purified enzyme was isolated by ion exchange HPLC. A 75×7.5 mm column (Biorad: Bio-GelTSK-IEX DEAE 5PW) was used to recover fractions with enzyme activity from isolated fractions obtained at a flow rate of 1 mL/min on a 0 to 400 mM NaCl linear gradient (50 mM Tris hydrochloric acid buffer, pH 7.0, 2 mM DTT), so as to purify the UDP-D-glucose:limonoid glucosyltransferase. During the purification, the enzyme was detected by adding enzyme to buffer (pH 7 to 8) containing 100 μM UDP-glucose and 30 μM nomilin labeled with radioactive isotope, bringing about a reaction for 15 to 30 minutes at 37° C., spotting the reaction product in silica gel thin layer chromatography, developing it with ethyl acetate:methyl ethyl ketone:formic acid:water (5:3:1:1), and checking to see whether or not the radioactivity was detected at the expected position based on the shift of the original nomilin from the starting point of the developing solvent to 0.88, and of the nomilin glucoside to 0.42 to confirm that limonoid glucosides had been produced as the reaction product.

The purified enzyme was further isolated by SDS-PAGE (polyacrylamide concentration of 17%, current of 24 mA), transferred to a PVDF membrane, and visualized by staining with Coumassie Brilliant Blue. The amino acid sequence on the amino-terminal side of the enzyme protein corresponding to a molecular weight of 56 to 58 kD was determined by Edman degradation, giving the following sequence.

| | |
|---|---|
| i) GTESLVHVLLVSF | (Sequence ID No. 3) |

The enzyme protein was treated by the Cleveland method, with isolation by SDS-PAGE (conditions: 24% acrylamide concentration; 25 mA current) of fragments partially digested by protease (S. aureus VI protease, by Sigma) in stacked gel, and subsequent transfer to PVDF membranes for visualization by staining with Coumassie Brilliant Blue. The amino acid sequences on the amino-terminal side of the two resulting peptide spots were determined by Edman degradation, giving the following interior sequences.

| | |
|---|---|
| ii) AGNFTYEPTPVGDG and | (Sequence ID No. 4) |
| iii) AEEAVADGGSSDR | (Sequence ID No. 5) |

2) Preparation of Primers

The following universal primers were designed on the basis of the above determined partial amino acid sequences i) (Sequence ID No. 3) and iii) (Sequence ID No. 5).

Sense Primer (Llg):

(Sequence ID No. 6)

GGNACNGA(a/g)(a/t)(g/c)N(c/t)TNGTNCA(c/t)GT

Antisense Primer (lgt14pr):

| | |
|---|---|
| CC(a/g)TCNGCNACNGC(t/c)TC | (Sequence ID No. 7) |

The primers having these sequences were then synthesized by the amidide method.* *Translator's note: Based on Japanese resources, the term “amidide” appears to correspond to “cyanoethyl phosphoramidite.”

3) Extraction of mRNA from Citrus unshiu (satsuma mandarin cv. Miyagawa wase)

Total RNA was extracted in the following manner using the SDS-phenol method from the fruit (albedo) of Citrus unshiu (satsuma mandarin cv. Miyagawa wase). The aforementioned albedo was frozen with liquid nitrogen and lyophilized. 0.5 g lyophilized albedo was frozen with liquid nitrogen, ground using a mortar and pestle that had been cooled with liquid nitrogen, and then immediately transferred to a mixture of 10 mL test buffer and 10 mL TE-buffered phenol. The mixture was stirred for 30 minutes at room temperature. The resulting mixture was centrifuged (5000×g, 20 minutes at 20° C.), and the aqueous phase was recovered. 10 mL test buffer was added to the remaining organic phase, and the material was stirred for 10 minutes at room temperature. This was centrifuged (5000×g, 20 minutes at 20° C.), and the aqueous phase was recovered. These operations were repeated, and the resulting aqueous phase was combined with that obtained earlier, resulting in 30 mL aqueous phase. 30 mL TE-buffered phenol was added, and the ingredients were stirred for 10 minutes at room temperature. The resulting mixture was centrifuged (5000×g, 20 minutes at 20° C.), and the aqueous phase was recovered. An equivalent amount of TE-buffered phenol was added to the aqueous phase, and the mixture was stirred for 10 minutes at room temperature. The resulting mixture was centrifuged (5000×g, 20 minutes at 20° C.), and the aqueous phase was again recovered. These operations were repeated. 2.75 mL of 5 M potassium acetate solution, 6.25 mL of iced ethanol, and 34 mL chloroform were added to the 25 mL aqueous phase that was ultimately obtained. The material was stirred for 30 minutes at room temperature and then centrifuged (5000×g, 20 minutes at 20° C.), and the aqueous phase was recovered. An equivalent amount of chloroform was added, the mixture was stirred for 10 minutes at room temperature and then centrifuged (5000×g, 20 minutes at 20° C.), the aqueous phase was recovered, 10 M lithium chloride solution was added to a final concentration of 3 M, and the material was allowed to stand over night at −20° C. before being centrifuged (20,000×g, 90 minutes at 4° C.). The precipitate was dissolved in 1 mL of water treated with DEPC, and 0.43 mL of 10 M lithium chloride solution was added. The mixture was allowed to stand over night at −20° C. before being centrifuged (18,000×g, 30 minutes at 4° C.). The precipitate was dissolved in 500 μL of DEPC water, 0.06-fold of 5 M lithium acetate and 2.5-fold iced ethanol were added to bring about precipitation, the material was allowed to stand over night at −20° C., and it was then precipitated (18,000×g, 30 minutes at 4° C.) to precipitate the RNA, giving total RNA.

mRNA was separated from the extracted total RNA in the following manner using Oligotex-dT30 <Super> (Takara). 1 mg of total RNA was dissolved in 200 μL test buffer, held for 5 minutes at 65° C., and then cooled on ice for 3 minutes. 40 μL of 5 M sodium chloride solution was added for 10 minutes of incubation at 37° C. The material was then centrifuged (18,000×g, 10 minutes at 20° C.), the precipitate was dissolved in 200 μL of DEP-treated water, and an equivalent amount of Oligotex-dT30 <Super> was added. 10 minutes of incubation at 65° C. was followed by precipitation with the addition of 0.1-fold of 3 M sodium acetate and 2.5-fold iced ethanol, the material was allowed to stand over night at −20° C., and it was then centrifuged (18,000×g, 30 minutes at 4° C.) to precipitate the RNA. The ethanol was removed, and the precipitate was dissolved in 50 μL of TE solution.

4) Cloning of Target Partial cDNA

The mRNA obtained in section 3) was used as template in reverse transcription with the Not I-d(T)18 primer (5′d[AACTGGAAGAATTCGCGGCCGCAGGAA(T)18]-3′) (Sequence ID No. 8) to prepare single-stranded cDNA. This was done in accordance with the instructions in the commercially available kit by Pharmacia (Ready-To-Go T-Primed First-Strand Kit). The 3′ end of all the single-stranded cDNA fragments thus obtained had a structure comprising the addition of the Not I adapter sequence (5′-TGGAAGAATTCGCGGCCGCAG-3′) (Sequence ID No. 9). The single-stranded cDNA was used as template in PCR using the sense and antisense primers synthesized in section 2) above. The PCR was carried out using a commercially available kit (Ampli Taq Gold, Perkin Elmer Applied Biosystems). The PCR was run for 35 cycles, where one cycle involved 60 seconds at 94° C. (denaturation), 60 seconds at 45° C. (annealing), and 120 seconds at 72° C. (extension).

Partial sequences of the cDNA coding for UDP-D-glucose:limonoid glucosyltransferase were obtained in the foregoing manner.

5) Preparation of cDNA Library from Citrus Albedo

5 μg of the mRNA purified in section 3) above was used as template in the synthesis of single-stranded cDNA with a linker primer (Oligo(dT)12-18) and reverse transcriptase derived from Moloney Murine Leukemia Virus (MMLV), and the second strand was synthesized with DNA polymerase. This was done using a commercially available kit (ZAP-cDNA Synthesis Kit by Stratagene). The ends of the double-stranded cDNA were smoothed with Pfu polymerase, and the EcoRI adapter was added using T4 ligase. The adapter was phosphorylated using T4 polynucleotide kinase and digested with XhoI, and 100 ng was ligated using T4 DNA ligase to a Lambda Uni-ZAP XR phagemid vector (Stratagene). This was packaged using a commercially available kit (Gigapack II packaging kit, Stratagene). A cDNA library was thus prepared

6) Screening of Target Full-length cDNA by Plaque Hybridization

From the cDNA library obtained in section 5) above, full-length cDNA was screened by plaque hybridization using as probe the partial cDNA obtained in section 4) above.

The cDNA library was first mixed with E. coli (line: XL1-Blue) and maintained for 15 minutes at 37° C. to allow the phages to be adsorbed by the cells, and plaques were formed on NZY precoated plates. Nylon membranes (Hybond-N+, Pharmacia) were attached to allow the phages to be transferred onto the membranes. The membranes were treated with alkali (0.5 N NaOH, 1.5 M NaCl) and then neutralized (1.0 M Tris pH 7.5, 1.5 M NaCl). The membranes were dried and were then fixed by UV cross-linking. Fixing was followed by hybridization, which was managed using a commercially available kit (DIG DNA labeling and detection kit, Boehringer Mannheim). Inserts were cut out from the clones selected by the screening (2×10⁴ pgu), and the inserts were base sequenced with an automated base sequencer (373A DNA Sequencing System, Perkin Elmer Applied Biosystems), revealing that the inserts were 1732 bp full-length cDNA coding for limonoid UDP-D-glucose:limonoid glucosyltransferase with 511 amino acid residues (estimated molecular weight 57 kDa).

The clones fully conserved the DNA sequences corresponding to the N terminal and interior amino acid sequences of Sequence ID Nos. 3, 4, and 5, and were thus designated CitLGT. The base sequence of CitLGT is given in Sequence ID No. 1, while the amino acid sequence encoded by CitLGT is given in Sequence ID No. 2.

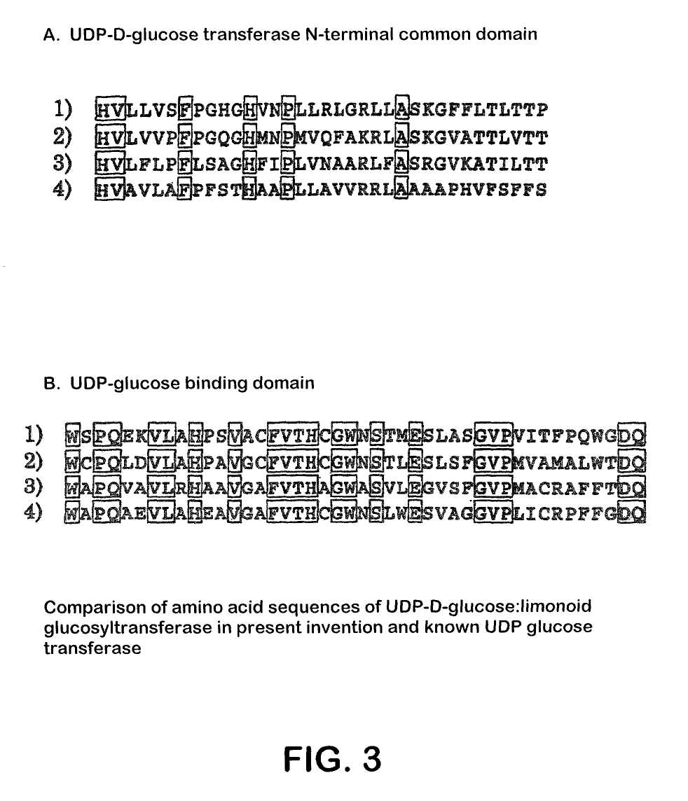

FIG. 3 gives the results of a comparison of the amino acid sequences of UDP-D-glucose transferases thus far isolated from plants and that of the UDP-D-glucose:limonoid glucosyltransferase in the present invention. In FIG. 3, the sequences of 1) through 4) are the amino acid sequences of A) the N-terminal site common domains and B) UDP glucose binding domains for, respectively, 1) the UDP-D-glucose:limonoid glucosyltransferase of the present invention (Sequence ID No. 2), 2) UDP-glucose:indole 3 acetate beta-D-glucosyltransferase (U81239 Arabidopsis thaliana), 3) solanidine UDP glucose glucosyltransferase (U82367 Solanum tuberosum), and 4) UDP-D-glucose:flavonoid 3-O-glucosyltransferase (AF000372 Vitis vinifera). Here, the sequence of A) the N terminal site common domain of the transferase of the present invention in 1) corresponds to the 8^{th} through 42^{nd} residues in Sequence ID No. 2, and the sequence of B) the UDP glucose binding domain corresponds to the 341^{st} through 384^{th} residues. The conserved residues are enclosed in the lines in the complete sequences of 1) through 4).

Despite the low homology in the complete amino acid sequences between the transferase of the present invention and the known UDP glucose transferases, the N terminal site common domain A) and the UDP glucose binding domain B) shown in FIG. 3 were conserved, indicating that the transferase of the present invention has the common characteristics of UDP glucose transferases.

Example 2

### Production of UDP-D-glucose:limonoid Glucosyltransferase in E. coli

1) Preparation of Expression Vectors and Transformants

The expression vector pExpLGT was obtained by splicing the coding sequence of the clones isolated in Example 1 downstream of the glutathione S transferase cDNA base sequence of pGEX4T- 1 (Pharmacia), a commercially available expression plasmid. The pExpLGT was then transformed with E. coli XL1-Blue (Stratagene). IPTG was then added to the medium so as to produce a fused protein comprising glutathione S transferase and UDP-D-glucose:limonoid glucosyltransferase.

2) Production of UDP-D-glucose:limonoid Glucosyltransferase

Clones of the aforementioned transformants were added to 2×YT medium (2×YT medium: 1.6% bactotryptone, 1.0% yeast extract, and 0.5% sodium chloride, pH 7.0), and ampicillin was added to a concentration of 100 μg/mL for 14 hours of shaking culture at 27° C. 1 mL of the resulting culture was added to 100 mL of 2×YT medium with ampicillin added to a concentration of 100 μg/mL, for another 10 hours of shaking culture at 27° C. Isopropyl thiogalactopyranosyl (IPTG) was added to a final concentration of 0.1 mM to the culture to induce protein fusion in 14 hours of shaking culture at 27° C.

The fused protein was then extracted from 100 mL of the resulting culture and treated with thrombin in accordance with the instructions of a commercial kit (GST Purification Module, Pharmacia), and was cleaved at the binding site with glutathione S transferase. 12.5% polyacrylamide gel electrophoresis (70 Vh, 15° C.) of the resulting purified UDP-D-glucose:limonoid glucosyltransferase fractions confirmed a band with the target molecular weight of about 57 kD. The purified enzyme fraction was separated by 12.5% polyacrylamide gel electrophoresis (70 Vh, 15° C.), and was transferred by Western blotting (25 mA, 15 minutes) to nitrocellulose membranes (Hybond-ECL, Pharmacia) for detection with monoclonal antibodies against the UDP-D-glucose:limonoid glucosyltransferase, resulting in an antigen-antibody reaction between the band assumed to be the enzyme and said antibodies. A commercially available kit (ECL Western blotting analysis system, Pharmacia) was used in accordance with the enclosed instructions for detection by Western blotting. It was thus confirmed that the transformants had produced UDP-D-glucose:limonoid glucosyltransferase.

### Example 3

### Cloning from Genome

### Isolation of Genomic DNA

1 g mature leaf was frozen with liquid nitrogen and ground with a mortar and pestle which had been pre-cooled with liquid nitrogen, and the ground material was transferred to 6 mL test buffer (100 mM Tris-HCl (pH 8.0), 50 mM EDTA, 0.5 M NaCl, 1% PVP (KT-30), 0.2% mercaptoethanol). 0.8 mL of 10% SDS was then added to the mixture, and the ingredients were mixed for 30 minutes at 68° C. 3 mL of 7.5 M ammonium acetate solution was then added and gently mixed, and the mixtures was allowed to stand for 30 minutes on ice. This was centrifuged (24,000×g, 4° C., 20 minutes), and the supernatant was recovered. This, too, was centrifuged (31,000×g, 4° C., 20 minutes), and the supernatant was again recovered. An equivalent amount (6 mL) of phenol-chloroform-isoamyl alcohol (25:24:1) was added to the supernatant, and the ingredients were gently stirred for 30 minutes at room temperature. The resulting mixture was centrifuged (24,000×g, 18° C., 15 minutes), and the aqueous phase was recovered. An equivalent amount of isopropanol was added to the aqueous phase and thoroughly mixed at room temperature. The mixture was allowed to stand for 30 minutes at room temperature, and then centrifuged (24,000×g, 18° C., 20 minutes). The supernatant was removed, the precipitate was air dried, 400 μL TE solution was added to the precipitate, and the material was held at 65° C. until the precipitate dissolved. RNase was added thereto, and the material was held for 5 minutes at 65° C., and then for 30 minutes at 37° C. Afterwards, 0.1-fold 3 M sodium acetate solution (40 μL) and 2.5-fold 99% ethanol (1 mL) were added and thoroughly mixed, and the mixture was then allowed to stand for 30 minutes. Afterwards, the material was centrifuged (18,000×g, 18c, 10 minutes) to bring about precipitation, the supernatant was removed, 1 mL of 70% ethanol was added to the precipitate, and the material was centrifuged (18,000×g, 18° C., 5 minutes) to precipitate the DNA, thus allowing genomic DNA to be recovered. The precipitated genomic DNA was centrifuged and dissolved in a suitable amount of TE solution.

2) Preparation of Primers

Primers for amplifying the codon region of the CitLGT sequence obtained in Example 1 were designed in the following manner.

Sense Primer (LGT-GF)

| | |
|---|---|
| ATGGGAACTGAATCTCTTGTTCAT | (Sequence ID No. 12) |

Antisense Primer (LGT-GR)

| | |
|---|---|
| TCAATACTGTACACGTGTCCGTCG | (Sequence ID No. 13) |

The primers having these sequences were then synthesized by the amidide method.

3) Cloning of Target Genomic Clones

PCR was carried out using the LGT-GF and LGT-GR prepared in section 2) above as primers and the genomic DNA obtained in section 1) above as template. The PCR was performed using a commercially available kit (Ampli Taq Gold, Perkin Elmer Applied Biosystems). The PCR was run for 30 cycles, where one cycle consisted of 60 seconds at 94° C. (denaturation), 60 seconds at 58° C. (annealing), and 120 seconds at 72° C. (extension).

75 μL of the PCR product was electrophoresed on 0.8% agarose gel, approximately 1.5 kb amplified fragments were recovered in accordance with the protocol of a commercially available kit (Gene Clean II, Bio 101), and were dissolved in 10 μL TE solution. The sample was introduced to the plasmid vector pCR 2.1 in accordance with the instructions in a commercially available kit (Original TA Cloning Kit, Invitrogen). The recombinant plasmid was transformed with E. coli XL1-Blue in the following manner. 5 μL recombinant plasmids were added to 100 μL competent cells prepared by the rubidium chloride method, and they were then allowed to stand for 30 minutes on ice. They were then held for 2 minutes at 42° C., and then immediately placed on ice again for 2 minutes. 1 mL of 2×YT medium was then added, and shaking culture was then performed for 1 hour at 37° C. After 1 hour, the contents were centrifuged (18,000×g, 4° C., 1 minute), 800 μL supernatant was removed and then resuspended, and 100 μL was used to inoculate LB agar medium (20 mg/L X-gal, 0.1 M IPTG, 50 mg/L ampicillin), which was spread with a spreader and cultured over night at 37° C. Following the culture, the isolated colonies were cultured again in 1 mL of LB medium (50 mg/L ampicillin), and the resulting culture was used as template in PCR with the LGT-GF and LGT-GR primers prepared in section 2) above. The PCR was run using a commercially available kit (Ampli Taq Gold, Perkin Elmer Applied Biosystems). The PCR was run for 30 cycles, where one cycle consisted of 60 seconds at 94° C. (denaturation), 60 seconds at 58° C. (annealing), and 120 seconds at 72° C. (extension). The reaction was carried out with 12.5 μL, and the amplified products were confirmed using 5 μL. The 7.5 μl remainder of the product of the clones confirmed as 1.5 kb products was treated with restriction enzymes, and the molecular polymorphism was searched, revealing the presence of clones with a different pattern than that of the CitLGT. The insert base sequence of the clones showing different molecular polymorphism was determined with an automatic sequencer (373A DNA Sequencing System, Perkin Elmer Applied Biosystems) using the sequence of the vector as a primer.

The DNA sequences corresponding to Sequence ID Nos. 3, 4, and 5 were fully conserved in the N terminal and interior amino acid sequences of the clone, which was designated CitLGT2. The base sequence of CitLGT2 is given in Sequence ID No. 10, and the amino acid sequence encoded by CitLGT2 is given in Sequence ID No. 11.

Residues 8 through 42 and 341 through 384 were the same as residues 8 through 42 (N-terminal common domain) and residues 341 through 384 (UDP glucose binding domain) in Sequence ID No. 2. The transferase of the present invention comprising the amino acid sequence in Sequence ID No. 11 was thus found to have the common characteristics of UDP glucose transferase in the same manner as in Example 1.

### Merit of the Invention

The present invention provides UDP-D-glucose:limonoid glucosyltransferase, DNA coding for UDP-D-glucose:limonoid glucosyltransferase, recombinant vectors comprising such DNA, transformants which have been transformed by such vectors, and methods for producing UDP-D-glucose:limonoid glucosyltransferase and limonoid glucosides.

Substances which are essential or important for preserving the quality and function of citrus fruits can thus be produced more efficiently.

### Sequence Listing

<110> Tohru Maotani, Director-General of National Institute of Fruit Tree Science, Ministry of Agriculture, Forestry and Fisheries

1. The recombinant protein of (a) or (b) below:
(a) protein comprising the amino acid sequence in Sequence ID No. 2;
(b) protein comprising amino acid sequence in Sequence ID No. 2 with one or more amino acid deletions, substitutions or additions, and having UDP-D-glucose:limonoid glucosyltransferase activity.

2. The recombinant protein of (a) or (b) below:
(a) protein comprising the amino acid sequence in Sequence ID No. 11;
(b) protein comprising amino acid sequence in Sequence ID No. 11 with one or more amino acid deletions, substitutions or additions, and having UDP-D-glucose:limonoid glucosyltransferase activity.

3. DNA coding for the recombinant protein of (a) or (b) below:
(a) protein comprising the amino acid sequence in Sequence ID No. 2;
(b) protein comprising amino acid sequence in Sequence ID No. 2 with one or more amino acid deletions, substitutions or additions, and having UDP-D-glucose:limonoid glucosyltransferase activity.

4. DNA according to claim 3, comprising the base sequence in Sequence ID No. 1.

5. DNA coding for the recombinant protein of (a) or (b) below:
(a) protein comprising the amino acid sequence in Sequence ID No. 11;
(b) protein comprising amino acid sequence in Sequence ID No. 11 with one or more amino acid deletions, substitutions or additions, and having UDP-D-glucose:limonoid glucosyltransferase activity.

6. DNA according to claim 5, comprising the base sequence in Sequence ID No. 10.

7. A recombinant vector comprising DNA selected from the group consisting of:
(a) DNA coding for the recombinant protein comprising the amino acid sequence in Sequence ID No. 2;
(b) DNA coding for the recombinant protein comprising the amino acid sequence in Sequence ID No. 2 with one or more amino acid deletions, substitutions or additions, and having UDP-D-glucose:limonoid glucosyltransferase activity;
(c) DNA of (a) or (b), comprising the base sequence in Sequence ID No. 1;
(d) DNA coding for the recombinant protein comprising the amino acid sequence in Sequence ID No. 11;
(e) DNA coding for the recombinant protein comprising the amino acid sequence in Sequence ID No. 11 with one or more amino acid deletions, substitutions or additions, and having UDP-D-glucose:limonoid glucosyltransferase activity;
(f) DNA of (d) or (e), comprising the base sequence in Sequence ID No. 10.

8. Transformants which have been transformed by a recombinant vector according to claim 7.

9. A method for producing UDP-D-glucose:limonoid glucosyltransferase, characterized in that transformants according to claim 8 are cultured in media, and UDP-D-glucose:limonoid glucosyltransferase is harvested from the resulting culture.

10. A method for producing limonoid glucosides, characterized in that transformants according to claim 8 are cultured in media, and limonoid glucosides are extracted from the resulting culture.
